(19) United States
(12) Patent Application Publication
(10) Pub. No.: US 2005/0136121 A1
(43) Pub. Date: Jun. 23, 2005

(54) Oral peptide delivery system with improved bioavailability

(75) Inventors: Alvin Kershman, Paradise Valley, MO (US); Jeff L. Shear, Chesterfield, MO (US)

Correspondence Address: GREENSFELDER HEMKER & GALE PC, SUITE 2000, 10 SOUTH BROADWAY, ST LOUIS, MO, 63102, US

(73) Assignee: SHEAR/KERSHMAN LABORATORIES, INC.

(21) Appl. No.: 11/013,751

(22) Filed: Dec. 16, 2004

Related U.S. Application Data
(60) Provisional application No. 60/531,821, filed on Dec. 22, 2003.

(51) Int. Cl. A61K 38/00; A61F 13/00

(52) U.S. Cl. 424/490; 514/2; 264/109

(57) ABSTRACT

An oral peptide delivery system where the peptide is present in a solid lipid suspension, wherein the suspension exhibits pseudotropic and/or thixotropic flow properties when melted, and in a preferred embodiment, the peptide is insulin, where the delivery system provides an improved bioavailability of the peptide.

### CROSS REFERENCE TO RELATED APPLICATION

This application claims the benefit of U.S. Provisional Patent Application entitled “Oral Peptide Delivery System with Improved Bioavailability”, Ser. No. 60/531,821, filed 22 Dec. 2003 by Alvin Kershman and Jeff L. Shear, which is herein incorporated by reference.

### FIELD OF THE INVENTION

The field of the invention is a delivery system for peptides that can be orally administered, more specifically, a delivery system for insulin that can be orally administered and provides improved bioavailablity.

### BACKGROUND OF THE INVENTION

The use of peptides and proteins, such as insulin, for the systemic treatment of certain diseases is now well accepted in medical practice. The role that the peptides play in replacement therapy is so important that many research activities are being directed towards the synthesis of large quantities by recombinant DNA technology. Many of these peptides are endogenous molecules which are very potent and specific in eliciting their biological actions.

A major factor limiting the usefulness of these substances for their intended application is that they are easily metabolized by plasma proteases when given parenterally. The oral route of administration of these substances, wherein the peptide is ingested, is even more problematic because in addition to proteolysis in the stomach, the gastric enzymes destroy them before they reach their intended target tissue. Any of the given peptides that survive passage through the stomach are further subjected to metabolism in the intestinal mucosa where a penetration barrier hinders entry into the cells.

The problems associated with oral or parenteral administration of proteins are well known in the pharmaceutical industry, and various strategies are being used in attempts to solve them. These strategies include incorporation of penetration enhancers, such as the salicylates, lipid-bile salt-mixed micelles, glycerides, and acylcarnitines, but these frequently are found to cause serious local toxicity problems, such as local irritation and toxicity, complete abrasion of the epithelial layer and inflammation of tissue. Other strategies to improve oral delivery include mixing the peptides with protease inhibitors, such as aprotinin, soybean trypsin inhibitor, and amastatin, in an attempt to limit degradation of the administered therapeutic agent. Unfortunately these protease inhibitors are not selective, and endogenous proteins are also inhibited. This effect is undesirable.

Insulin is the mainstay for treatment of virtually all Type-I and many Type-II diabetic patients. When necessary, insulin may be administered intravenously or intramuscularly; however, long-term treatment relies on subcutaneous injection. Subcutaneous administration of insulin differs from physiological secretion of insulin in at least two major ways. First, the kinetics of absorption are relatively slow and thus do not mimic the normal rapid rise and decline of insulin secretion in response to ingestion of food, and second, the insulin diffuses into the peripheral circulation instead of being released into the portal circulation. The preferential effect of secreted insulin on the liver is thus eliminated. Nonetheless, such treatment has achieved considerable success.

Preparations of insulin can be classified according to their duration of action into short-, intermediate-, or long-acting and by their species or origin—human, porcine, bovine, or a mixture of bovine and porcine. Human insulin is now widely available as a result of its production by recombinant DNA techniques.

Attempts have been made to administer insulin orally, nasally, rectally, and by subcutaneous implantation of pellets. Although oral delivery of insulin would be preferred by patients and would provide higher relative concentrations of insulin in the portal circulation, attempts to increase intestinal absorption of the hormone have met with only limited success. Efforts have focused on protection of insulin by encapsulation or incorporation into liposomes. See, generally, Goodman and Gilman, the Pharmacological Bases of Therapeutics (8th Ed.), pages 1463-1495, McGraw-Hill, NY (1993).

The present delivery system provides an orally administrated peptide which is readily absorbed in the intestine, which delivers the peptide into the bloodstream and provides improved bioavailability.

### SUMMARY OF THE INVENTION

The present invention is an oral peptide delivery system, wherein the peptide is ingested, providing improved bioavailability comprising at least one lipid and dry particles, wherein, the dry particles contain at least one peptide and at least one filler. The dry particles are continuously coated with the lipid and form a homogenous suspension with the lipid. The suspension exhibits pseudoplastic and/or thixotropic properties, and the suspension is formed or shaped into the appropriate solid dosage form by molding or pouring the suspension when in a liquid or semi-liquid state.

The present invention further includes a method for preparing an oral peptide delivery system comprising the steps of melting at least one lipid, and dry-mixing dry particles comprising at least one filler and at least one peptide. The dry particles are mixed with the melted lipid to form a suspension such that the dry particles are continuously coated by the lipid such that the suspension exhibits pseudoplastic and/or thixotropic properties. The suspension is poured or molded into a dosage form.

In a second embodiment of the invention, the oral peptide delivery system is a suspension in a liquid lipid system wherein the dry particles contain at least one peptide and at least one filler.

### DETAILED DESCRIPTION OF THE INVENTION

As used herein the term “insulin” refers to any of the various insulins that are known. Insulins are divided into three categories according to promptness, duration and intensity of action following subcutaneous administration, i.e., as mentioned above, rapid, intermediate or long-acting. Crystalline regular insulin is prepared by precipitation in the presence of zinc chloride and modified forms have been developed to alter the pattern of activity. The extended and prompt insulin-zinc suspensions are also contemplated for use in the invention. The insulin can be, for example, of human, bovine, ovine or other animal origin or can be a recombinant product.

Short- or rapid-acting insulins are simply solutions of regular, crystalline zinc insulin (insulin injection) dissolved in a buffer at neutral pH. These have the most rapid onset of action but the shortest duration, i.e., glucose levels reach a low point within 20-30 minutes and return to baseline in about 2-3 hours. Intermediate-acting insulins are formulated so that they dissolve more gradually when administered subcutaneously; their durations of action are thus longer. The two preparations most frequently used are neutral protamine Hagedom (NPH) insulin (isophane insulin suspension) and Lente insulin (insulin zinc suspension).

As used herein, the term insulin is also contemplated to encompass insulin analogs. A recent development of insulin with altered rates of absorption has raised interest. Insulin with aspartate and glutamate substituted at positions B9 and B27, respectively, crystallizes poorly and has been termed “monomeric insulin”. This insulin is absorbed more rapidly from subcutaneous depots and thus may be useful in meeting postprandial demands. By contrast, other insulin analogs tend to crystallize at the site of injection and are absorbed more slowly. Insulins with enhanced potency have been produced by substitution of aspartate for histidine at position B10 and by modification of the carboxyl-terminal residues of the B chain.

While the ensuing description is primarily and illustratively directed to the use of insulin as a peptide component in various compositions and formulations of the invention, it will be appreciated that the utility of the invention is not limited to the following peptide species: calcitonin, ACTH, glucagon, somatostatin, somatotropin, somatomedin, parathyroid hormone, erythropoietin, hypothalmic releasing factors, prolactin, thyroid stimulating hormone, endorphins, antibodies, hemoglobin, soluble CD-4, clotting factors, tissue plasminogen activator, enkephalins, vasopressin, non-naturally occurring opioids, superoxide dismutase, interferon, asparaginase, arginase, arginine deaminease, adenosine deaminase ribonuclease, trypsin, chemotrypsin, and papain, alkaline phosphatase, and other suitable enzymes, hormones, proteins, polypeptides, enzyme-protein conjugates, antibody-hapten conjugates, viral epitopes, etc. Peptide derivatives and polypeptides contemplated in this invention are further disclosed in U.S. Pat. No. 6,770,625, which is hereby incorporated by reference.

In one embodiment of the present invention, the delivery system is a solid lipid suspension. The solid lipids of the present invention may be of animal, vegetable or mineral origin, which are substantially water-insoluble, inert, non-toxic hydrocarbon fats and oils and derivatives thereof, and may comprise any of the commonly commercially available fats or oils approved by the Food & Drug Administration, having melting points in the range of about 90 to 160° F. (32 to 71° C.). The lipid may comprise a vegetable oil base commonly known as hard butter. Hard butters are hydrogenated, press fractionated, or other processed oils that are processed or recombined to have a solid fat index (percent solid fat vs. temperature) similar to that of cocoa butter. However, other lipids may be used that are relatively hard or solid at room temperature, but melt rapidly in the mouth at a temperature of about 92° to 98° F. (29 to 32° C.)(mouth temperature). The lipid is employed in the amounts within the range of from about 20 to 50%. Above about 50%, the suspension flows too readily and does not exhibit thixotropic or pseudoplastic flow properties. When present below about 20%, the amount of lipid is not sufficient to completely coat the dry particles.

In a second embodiment of the present invention, the lipid is a liquid. Examples of suitable lipids include tallow, hydrogenated tallow, hydrogenated vegetable oil, almond oil, coconut oil, corn oil, cottonseed oil, light liquid petrolatum, heavy liquid petrolatum, olein, olive oil, palm oil, peanut oil, persic oil, sesame oil, soybean oil or safflower oil. In this embodiment, fatty acids are also considered suitable, such as palmitic acid and linoleic acid.

Additionally, stearines can be used as a lipid in the present invention. The addition of stearines to the solid lipids provides the favorable property of mold-release. Further, the addition of stearines raises the melting point of the composition as high as about 100° F. (38° C.), which is particularly beneficial when the product is shipped or stored in unrefrigerated compartments.

The fillers of the present invention are pharmacologically inert and optionally nutritionally beneficial to humans and animals. Such fillers include cellulose such as microcrystalline cellulose, grain starches such as cornstarch, tapioca, dextrin, sugars and sugar alcohols such as sucrose sorbitol, xylitol, mannitol and the like. Preferred fillers include non-fat milk powder, whey, grain brans such as oat bran, and fruit and vegetable pulps. Preferred fillers are finely divided and have a preferred average particle size in the range of about 0.10 to 500 microns. The fillers are present in the drug delivery device in a concentration of about 50 to 80%. Optionally, the peptide particles can also serve as filler in the delivery system.

Optionally, an emulsifier or surfactant may be used in the lipid suspension. Any emulsifier or surfactant approved for use in foods by the Food and Drug Administration and having a relatively low HLB value, in the range of about 1 to 3, is suitable for use in the present invention. The appropriate surfactant minimizes the surface tension of the lipid, allowing it to oil wet and encapsulate the non-oil solid particles. Typically, the surfactant is present in the delivery system in the concentration of about 0.1 to 1.0%. Suitable surfactants include alkyl aryl sulfonate, alkyl sulfonates, sulfonated amides or amines, sulfated or sulfonated esters or ethers, alkyl sulfonates, of dioctyl sulfonosuccinate and the like, a hydrated aluminum silicate such as bentonite or kaolin, triglycerol monostearate, triglycerol monoshortening, monodiglyceride propylene glycol, octaglycerol monooleate, octaglycerol monostearate, and decaglycerol decaoleate. The preferred surfactant is lecithin.

In a preferred embodiment, the polypeptide is microencapsulated. Such microencapsulation includes sustained release encapsulation. Any known method of encapsulation is suitable in the present invention. Such methods include, but are not limited to air coating, chemical erosion, coacervation, fluid bed coating, macroencapsulation, microencapsulation, osmosis, pan spray coating, physical erosion, polymer protein conjugate systems, and polymeric microspheres. A preferred method involves slowly blending the drug with a filming agent solution to form granulated particles. The granulated particles are allowed to dry on a tray and are sieved to the desired size, typically in the range of from about 200 to 500 microns. The coating materials include, but are not limited to, acrylic polymers and co-polymers, alginates, calcium stearate, cellulose, including methylcellulose, ethylcellulose, and hydroxypropyl cellulose, gelatins, glyceryl behenate, glycholic acid and its various forms, ion exchange resins, lactic acid and its various forms, lipids, methacrylic monomers, methacrylic polymers and co-polymers, polyethylene glycol polymers, shellac (pharmaceutical glaze), stearic acid, glycerol esters of fatty acids and waxes.

In a second embodiment, the peptide is suspended in the lipid as dry particles, and the resulting dosage form is microencapsulated, so that not only the peptide, but the lipid and other dry particles are microencapsulated. In a third embodiment, the lipid formulation is enclosed in a gel capsule, and the capsule is coated with a coating material for encapsulation.

In another embodiment of the present invention, the peptide is not microencapsulated, but suspended in the lipid as dry particles. Typically the peptide is present in the delivery device in a concentration of 30% or less. However, the peptide can comprise all of the dried particles, to provide the necessary dose.

Optionally, the dry particles include flavorings that make the device taste and smell appealing to humans or animals. The flavorings can be natural or synthetic, and can include fruit flavorings, citrus, meat, chocolate, vanilla, fish, butter, milk, cream, egg or cheese. The flavorings are typically present in the device in the range of about 0.05 to 50.0%.

The delivery device may also include other pharmaceutically acceptable agents, such as sweetening agents, including hydrogenated starch hydrolysates, synthetic sweeteners such as sorbitol, xylitol, saccharin salts, L-aspartyl-L-phenylalanine methyl ester, as well as coloring agents, other binding agents, lubricants, such as calcium stearate, stearic acid, magnesium stearate, antioxidants such as butylated hydroxy toluene, antiflatuants such as simethicone and the like. Additional agents include protease inhibitors, absorption enhancers and mucoadhesives.

Optionally, rupturing agents are used to rapidly deliver the peptide into the recipient's system. A typical rupturing agent is a starch that swells in the presence of water. Various modified starches, such as carboxymethyl starch, currently marketed under the trade name Explotab or Primojel are used as rupturing agents. A preferred rupturing agent is sodium starch glycolate. When ingested, the capsule or pellet swells in the presence of gastric juices and ruptures.

In one embodiment of the present invention, the rupturing agent is present inside the microcapsule. As water penetrates the microcapsule, it swells the starch and ruptures the capsule, rapidly delivering the peptide to the system. Additional rupturing agents are disclosed in U.S. Pat. No. 5,567,439, which is hereby incorporated by reference.

In another embodiment, the rupturing agent is present in the lipid suspension, which ruptures the pellet, but leaves the microcapsules intact. This allows the delayed delivery of the drug farther along in the digestive system, in the intestines or the colon. The present invention is particularly effective in this embodiment, in that the ingested pellet may be chewable, where the pellet cleaves in the lipid suspension when chewed, but leaves the microcapsules intact. Tablets or gel capsules, when chewed, typically result in damage to or rupturing of the microcapsules defeating the effectiveness of the microcapsules.

In yet another embodiment, multiple drugs have multiple encapsulations, each containing an rupturing agent. The filming agents used for encapsulation are selected to disintegrate at selected pH conditions, which rupture and release each peptide at desired locations in the digestive system. In another embodiment, the use of a mucoadhesive could effect the delivery of the peptide to the colon.

The process for preparing the above delivery system comprises melting the lipid and mixing with the surfactant. The dry particles are mixed with the melted lipid mixture to form a suspension exhibiting pseudoplastic and/or thixotropic flow properties, and poured or molded to provide dosage forms.

The dry particles, which include the peptide, filler and optional flavorings and additives, are pre-blended and typically have a particle size in the range of from about 50 to 450 microns. The pre-blended particles are gradually added to the heated lipid base until a high solid suspension is obtained, typically in the range of about 50 to 80% particles and from about 50 to 20% lipid. The preferred form of peptide is micronized peptide.

Slow addition of the dry particles is critical in the production of the device, to insure that the particles are suspended in their micronized state and not as agglomerated clumps. Moreover, rapid addition can cause the mixing process to fail in that the melted suspension will not have the desired flow properties, but instead will be a granular oily mass (a sign of product failure). The mixing step is accomplished in a heated mixing device that insures thorough mixing of all materials with minimal shear, such as a planetary mixer or a scrape surface mixer. After the suspension is formed, the product is poured into molds and allowed to cool. De-molding and packaging are then performed. Alternatively, the suspension can be super-cooled and sheeted in a semi-soft format. The sheet is processed through forming rolls containing a design or configuration that embosses and forms the final shape. Liquid lipid suspensions can be placed in gel capsules as dosage forms.

The following examples are to illustrate the claimed invention and are not intended to limit the claims in any way. All of the percentages are by weight unless otherwise indicated.

### EXAMPLES

The Examples and control were prepared according to the following procedure.

Forming the Suspension

The lipid (kaomel) was heated in a Hobart 5 Quart planetary mixer jacketed with a heating mantle in the range of about 140 to 150° F. (60 to 66° C.) and melted. The surfactant, lecithin, was added to the lipid with mixing, and the mixture was allowed to cool to about 135° F. (58° C.).

The dry particles, including the peptide (insulin), the fillers which included cls-555 (a partially hydrogenated vegetable oil), cocoa, eudragit L 100 (a methacrylic acid copolymer coating for the insulin) and avicel ph 102 (a cellulose disintegrating agent), and the flavorings (milk flavor, salt, vanilla, sucralose, milk crumb, fudge flavor, magnasweet and choc enhancer) were screened to a particle size in the range of about 200 and 500 microns and dry-blended. The dry particles were slowly added incrementally to the lipid/surfactant mixture with mixing over a period of about 1 hour, to provide a smooth suspension with no lumps or agglomerations. The suspension exhibited thixotropic and pseudoplastic flow properties. It was molded and cooled to about 70° F. (21° C.). The suspension shrank as it cooled, and easily released from the mold when inverted.

### Study 1

### Examples 1 and 2, and Control 1

Two insulin preparations and one control lipid suspension without insulin were fed to mice. Examples 1 and 2 were solid lipid suspensions containing insulin. Example 1 (see Table 1) was formulated with granulated human recombinant insulin, and Example 2 (see Table 2) was formulated with coated human recombinant insulin. Each of the two insulin preparations provided 10 micrograms of insulin to each mouse, which is 0.24 units, with each 20 mg dose. Control 1 (See Table 3) was a solid lipid suspension with no insulin, and was fed to the mice. Food was provided ad libitum during the evaluation.

### Example 1

### Forming a Suspension of Insulin

TABLE 1

| Ingredient | Weight % |
|---|---|
| kaomel (lipid) | 60.0 |
| clsp555 | — |
| Eudragit L 100 (cellulose filler) | 5.92 |
| avicel ph (filler) | 20.0 |
| cocoa (filler, flavor) | 11.0 |
| Lecithin (surfactant) | 0.60 |
| Salt | 0.25 |
| milk flavor | 1.00 |
| Insulin (peptide) | 0.05 |
| sucralose | 0.10 |
| milk crumb | 0.25 |
| fudge flavor | 0.20 |
| fna vanilla | 0.25 |
| magnasweet | 0.13 |
| choc enhancer | 0.25 |
| Totals | 100.0 |

### Example 2

### Forming a Suspension of Microencapsulated Insulin

TABLE 2

| Ingredient | Weight % |
|---|---|
| kaomel (lipid) | 60.0 |
| clsp555 | — |
| Eudragit L 100 (cellulose filler) | 5.84 |
| avicel ph (filler) | 20.0 |
| cocoa (filler, flavor) | 11.0 |
| Lecithin (surfactant) | 0.60 |
| Salt | 0.25 |
| milk flavor | 1.00 |
| Insulin, coated (peptide) | 0.135 |
| sucralose | 0.10 |
| milk crumb | 0.25 |
| fudge flavor | 0.20 |
| fna vanilla | 0.25 |
| magnasweet | 0.13 |
| choc enhancer | 0.25 |
| Totals | 100.0 |

### Control 1

### Forming a Suspension With No Insulin

TABLE 3

| Ingredient | % |
|---|---|
| kaomel (lipid) | 40.0 |
| clsp555 | 20.0 |
| Eudragit L 100 (cellulose filler) | 5.0 |
| avicel ph (filler) | 20.0 |
| cocoa (filler, flavor) | 10.0 |
| Lecithin (surfactant) | 0.6 |
| Salt | 0.25 |
| milk flavor | 1.0 |
| Insulin (peptide) | — |
| sucralose | 0.1 |
| milk crumb | 0.25 |
| fudge flavor | 0.2 |
| fna vanilla | 0.25 |
| magnasweet | 0.13 |
| choc enhancer | 0.25 |
| Totals | 100.0 |

Results:

The lipid suspension with coated insulin (Example 2) gave a reduced serum glucose level of about 20%, indicating that oral insulin had been delivered into the blood stream. The glucose level remained reduced until about 90 minutes had lapsed from administration. At about 120 minutes, the glucose level returned to normal. Insulin levels increased for both Examples 1 and 2, but did not increase significantly for Control 1.

### Study 2

### Examples 3-5

Female adult dogs were fed the formulations from Examples 3 to 5. Further dogs were injected with Humulin and given 0.05 mL of 100 U/mL. Before being fed or injected the formulations, the animals fasted overnight. The dogs were fed 4 to 6 hours after dosing.

### Example 3

### Coated Insulin, pH 6

TABLE 4
Batch Formula

| Ingredient | Weight % |
|---|---|
| Kaomel (lipid) | 72.8 |
| Eudragit L 100 (cellulose filler) | 5.0 |
| Avicel ph (filler) | 20.0 |
| Lecithin (surfactant) | 1.0 |
| Insulin, coated (pH 6) | 1.2 |
| Total | 100.0 |

The same formulation was used as for Example 3, however, uncoated insulin was used in place of coated insulin.

### Example 5

### Uncoated Insulin in Coated Pellets

The same formulation was used as for Example 4, however, the final pellet formulation was coated with the coating material (pH 6) of Example 3.

### Results

The dogs were fed the formulations of Examples 3, 4 and 5 had no significant increase in levels of glucose, e.g., their serum glucose levels were constant. When the dogs were fed dog food 4 to 6 hours after dosing, their glucose levels remained unchanged.

The present drug delivery system embodied in Examples 3, 4 and 5 maintained an essentially constant serum glucose level after administration of the insulin, and after eating dog food. This could indicate a steady release of insulin into the blood stream from the lipid delivery system.

When the dogs were injected with Humulin, their glucose levels dropped significantly (67%) within minutes, and after about 2 hours following dosing, the glucose level began increasing. The dogs ate dog food, at 4 to 6 hours. At hours 6 to 10, the glucose levels of the dogs were above the normal levels of glucose. After eating, the glucose levels returned to normal levels at about 12 hours.

The injected Humulin resulted in dramatic swings in serum glucose levels. There was an initial drop in serum glucose following injection, followed by an increase in serum glucose *after the dogs ate dog food. The serum glucose levels leveled out after 12 hours.

1. A method for preparing an oral peptide delivery system comprising the steps of:
melting at least one lipid;
dry-mixing dry particles comprising at least one filler and at least one peptide;
mixing the dry particles with said melted lipid to form a suspension such that said dry particles are continuously coated by said lipid such that said suspension exhibits pseudoplastic and/or thixotropic properties, and pouring or molding said suspension into a dosage form.

2. The method of claim 1 in which at least some of said peptide particles are microencapsulated with a filming agent.

3. The method of claim 2 in which said microencapsulated peptide particles are micronized.

4. The method of claim 2 in which said peptide particles are microencapsulated with a rupturing agent.

5. The method of claim 4 in which said rupturing agent is sodium starch glycolate.

6. The method of claim 5 in which said lipid source forms 20% to 40% by weight of said suspension, and said dry particles form 60% to 80% by weight of said suspension.

7. The method of claim 6 in which said fillers have a size ranging from 10 to 500 microns in diameter and comprise whey.

8. The method of claim 1 in which said lipid is selected from the group consisting of a hard butter, petroleum wax, vegetable fat or animal stearines.

9. The method of claim 8 in which said lipid suspension contains a rupturing agent.

10. The method of claim 9 in which said rupturing agent is sodium starch glycolate.

11. The method of claim 10 in which the dry particles include artificial flavorings and/or surfactants.

12. An oral peptide delivery system comprising:
A. at least one lipid; and
B. dry particles;
wherein, the dry particles contain at least one peptide and at least one filler;
wherein, the dry particles are continuously coated with the lipid and form a homogenous suspension with the lipid;
wherein the suspension exhibits pseudoplastic and/or thixotropic properties; and
wherein the suspension is formed or shaped into the appropriate solid dosage form by molding or pouring the suspension when in a liquid or semi-liquid state.

13. The peptide delivery system of claim 12 in which at least part of the peptide is microencapsulated.

14. The peptide delivery system of claim 13 in which said microencapsulated peptide contains a rupturing agent.

15. An oral peptide delivery system comprising:
A. at least one lipid; and
B. dry particles
wherein the dry particles contain at least one peptide and at least one filler;
wherein the dry particles are continuously coated with the lipid and form a homogenous suspension with the lipid;
wherein the suspension exhibits pseudoplastic and/or thixotropic properties;
wherein the suspension is formed or shaped into the appropriate solid dosage form by molding or pouring the suspension when in a liquid or semi-liquid state;
wherein at least part of said peptide particles is present in said suspension as a microencapsulated particle.

16. The delivery system of claim 15 in which said microencapsulated peptide contain therein a rupturing agent.

17. The peptide delivery system of claim 16 in which said rupturing agent comprises sodium starch glycolate.

18. The oral peptide delivery system of claim 12, wherein the peptide is insulin.

19. The oral peptide delivery system of claim 12, wherein the system includes additional drugs, medicaments, surfactants or food supplements.

20. The oral peptide delivery system of claim 12, wherein the filler comprises the peptide.

21. The oral peptide delivery system of claim 12, wherein the system includes a mucoadhesive.

22. A method for preparing an oral peptide delivery system comprising:
dry-mixing dry particles containing at least one peptide and at least one filler;
adding the dry particles to a liquid lipid;
forming a homogeneous suspension wherein the dry particles are continuously coated with the lipid;
and forming into the appropriate dosage form.
